(19) United States
(12) Patent Application Publication
(10) Pub. No.: US 2006/0286052 A1
(43) Pub. Date: Dec. 21, 2006

(54) Esterification product and cosmetic

(76) Inventors: Jun Oki, Izumiotsu-shi (JP); Yasunori Noguchi, Izumiotsu-shi (JP)

Correspondence Address: BIRCH STEWART KOLASCH & BIRCH, PO BOX 747, FALLS CHURCH, VA, 22040-0747, US

(21) Appl. No.: 10/568,779

(22) PCT Filed: Aug. 31, 2004
(86) PCT No.: PCT/JP04/13079

§ 371 Date: Feb. 21, 2006

(30) Foreign Application Priority Data
Sep. 5, 2003 (JP) 2003-313715

(51) Int. Cl. A61K 8/37; C07C 57/02

(52) U.S. Cl. 424/70.1; 554/227

(57) ABSTRACT

An oily raw material which is usable as an oily raw material for cosmetics and has water-holding properties; and a cosmetic containing this material. The water-holding oily raw material is an esterification product obtained by esterifying polyglycerol having an average degree of polymerization of 6 to 15 with a C8-22 branched fatty acid. This esterification product preferably has a degree of esterification of 60% or higher. This esterification product can be used to produce a cosmetic.

### TECHNICAL FIELD

The present invention relates to esterification products, and particularly to fatty acid esters of polyglycerol obtained by esterifying polygricerol and fatty acid and to a cosmetic including the ester.

### BACKGROUND ART

Oily raw materials are employed as major raw materials of a wide variety of cosmetics and are used as important ingredients of cream and lotion. Oily raw materials employed for cosmetics are used for purposes such as inhibition of moisture transpiration from the skin and improvement in feeling in use. Oily raw materials include natural oily components such as vegetable oils and animal oils; higher alcohol or higher fatty acid purified and isolated from oils, fats and waxes; hydrocarbon oils obtained by purification by fractional distillation of petroleum; and synthesized ester oils.

As one of the animal oils, lanolin is known, which is obtained by purification of oils and fats adhering on wool. Lanolin is a useful oily raw material having a high water-holding property not available in other oils and fats used for oily raw materials. It has been recognized for a long time that lanolin is a useful oily raw material. Lanolin has been used widely for all types of cosmetics such as skin car cosmetics, hair care cosmetics and makeup cosmetics.

As ester oils, known are diglycerol branched fatty acid esters such as diglyceryl diisostearate and diglyceryl triisostearate; and malic acid branched fatty acid esters such as diisostearyl malate. These esters are produced using fatty acids derived from vegetable oils and they are known as oily raw materials having water-holding properties like lanolin. In addition, these esters are used for various types of cosmetics, particularly for labia cosmetics such as lipstick. Besides these esters, as an ester which is produced by using a fatty acid derived from vegetable oil and which is used as an oily raw material of cosmetics, decaglyceryl pentaisostearate is known.

As still other conventional oily raw materials, water-holding oily raw materials are disclosed as water-holding oils in, for example, Patent Japanese Patent Laid-Open No. 11-246354 and Japanese Patent Laid-Open No. 2002-255738. In [0021] of Japanese Patent Laid-Open No. 11-246354 and [0010] of Japanese Patent Laid-Open No. 2002-255738, lanolin derivatives; substances obtained by modifying lanolin derivatives with polyoxyalkylene; amino acid-based ester oils; polyhydric alcohol fatty acid esters; polyglycerol fatty acid esters; and hydroxy fatty acid esters are disclosed as oily raw materials having water-holding properties.

### DISCLOSURE OF INVENTION

Because of animal protection growing today in Europe and the United States, use of lanolin derived from animal oils is coming to be avoided by consumers. Use of oily raw materials derived from beef tallow, which is one of animal oils other than lanolin, is avoided more by consumers due to legal restriction on the use and the mad cow disease problem in these days. Under such circumstances where beef fallow is avoided, use of lanolin derived from sheep, which sometimes suffer from mad sheep disease (scrapie) like mad cow disease, has been avoided by the cosmetic industry.

On the other hand, diglycerol branched fatty acid esters and malic acid branched fatty acid esters produced using fatty acids derived from plants exert water-holding properties, but the water-holding properties are insufficient in comparison to lanolin. According to examination of the water-holding property of conventional decaglyceryl pentaisostearate, which is produced by using a plant-derived fatty acid, the water-holding property is inferior to that of lanolin. In addition, decaglyceryl pentaisostearate emits odor and exerts an insufficient moisture retention property when being used for cosmetics.

Other oily raw materials disclosed concretely in Japanese Patent Laid-Open No. 11-246354 and Japanese Patent Laid-Open No. 2002-255738 exert only water-holding properties insufficient in comparison to lanolin.

In view of the above-mentioned circumstances, an object of the present invention is to provide an esterification product that is an oily raw material which can be produced using a plant-derived fatty acid which is preferred by consumers and that exerts a water-holding property and also exerts a moisture retention property when being used as a raw material of a cosmetic.

In addition, another object of the present invention is to provide a cosmetic including an esterification product having a water-holding property and a moisture retention property.

The present inventors studied diligently in order to solve the aforementioned problems and, as a result, have accomplished the present invention by founding that an esterification product of fatty acid which is obtained by esterifying a specific polyglycerol and a specific branched fatty acid and which has a predetermined degree of esterification shows a water holding property and is an esterification product suitable for water holding applications.

That is, the present invention provides an esterification product which is an esterification product obtained by esterifying polyglycerol and a fatty acid, wherein the polyglycerol has an average degree of polymerization of from 6 to 15 calculated on the basis of the hydroxyl value thereof, the fatty acid is at least one branched fatty acid selected from branched fatty acids having 8 to 22 carbon atoms, the esterification product has a degree of esterification of 60% or more. By the esterification product as used herein is meant both an esterification product including a free fatty acid and an esterification product including no free fatty acid.

The average degree of polymerization (n) calculated on the basis of a hydroxyl value is a value calculated by terminal analysis and is calculated from the following (Formula 1) and (Formula 2).
Molecular weight=74n+18 (Formula 1)
Hydroxyl value=56110(n+2)/molecular weight (Formula 2)

The hydroxyl value is a value which is used as an indicator of the size of the number of hydroxyl groups contained in an esterification product. It is the number of milligram of potassium hydroxide needed to acetylate free hydroxyl groups contained in 1 g of the esterification product. The number of milligram of potassium hydroxide is calculated according to “Standard Methods for the Analysis of Fats, Oils and Related Materials (I) set by Japan Oil Chemists' Society (1996)” edited by Japan Oil Chemists' Society.

The degree of esterification is a value calculated from {M/(n+2)}×100=degree of esterification (%), wherein the average degree of polymerization of polyglycerol calculated on the basis of a hydroxyl value is n, the number of hydroxyl groups the polyglycerol has is n+2, and the molar number of branched fatty acid added is M.

The esterification product is desirably an esterification product having an acid value of 3.0 or less. The acid value is a value which is used as an indicator of the size of free fatty acid contained in an esterification product. It is the number of milligram of potassium hydroxide needed to neutralize free fatty acid contained in 1 g of the esterification product, which is calculated according to “Standard Methods for the Analysis of Fats, Oils and Related Materials set by Japan Oil Chemists' Society (1996)” edited by Japan Oil Chemists' Society.

The present invention is directed to a cosmetic including the esterification product.

The esterification product exerts a water-holding property and, when being used as a raw material of a cosmetic, causes the cosmetic to be a cosmetic having a good moisture retention property because it has a constitution where a specific polyglycerol and a specific branched fatty acid have been esterified and a degree of esterification of 60% or more has been established. When the esterification product has an acid value of 3.0 or less, it is an esterification product superior in odor.

A cosmetic constituted as mentioned above is a cosmetic produced by inclusion of the esterification produce of the present invention and, therefore, is a cosmetic excellent in water-holding property and moisture retention property.

### BEST MODE FOR CARRYING OUT THE INVENTION

The present invention is explained below in detail with reference to embodiments. The esterification product of a branched fatty acid according to the present invention is an esterification product of a branched fatty acid obtained by esterifying hydroxyl groups which polyglycerol has with one or more kinds of specific branched fatty acid.

As the polyglycerol, a polyglycerol having an average degree of polymerization, calculated on the basis of a hydroxyl value, of from 6 to 15, preferably from 8 to 12 is selected.

As the specific branched fatty acid, one or more kinds of fatty acid having from 8 to 22, preferably from 14 to 20, carbon atoms are selected. Use of a saturated branched fatty acid is preferred. Examples of saturated branched fatty acids having from 8 to 22 carbon atoms include 2-ethylhexanoic acid, 3,5,5-trimethylhexanoic acid, 4-propylpentanoic acid, 4-ethylpentanoic acid, 2-methyldecanoic acid, 3-methyldecanoic acid, 4-methyldecanoic acid, 5-methyldecanoic acid, 6-methyldecanoic acid, 7-methyldecanoic acid, 9-methyldecanoic acid, 6-ethylnonanoic acid, 5-propyloctanoic acid, 3-methylundecanoic acid, 6-propylnonanoic acid, 2-methyldodecanoic acid, 3-methyldodecanoic acid, 4-methyldodecanoic acid, 5-methyldodecanoic acid, 11-methyldodecanoic acid, 7-propyldecanoic acid, t2-methyltridecanoic acid, 12-methyltridecanoic acid, 2-methyltetradecanoic acid, 4-methyltetradecanoic acid, 13-methyltetradecanoic acid, 14-methylpentadecanoic acid, 2-ethyltetradecanoic acid, 15-methylhexadecanoic acid, 2-propyldecanoic acid, 2-ethylhexadecanoic acid, 14-ethylhexadecanoic acid, 14-methylheptadecanoic acid, 15-methylheptadecanoic acid, 16-methylheptadecanoic acid, 2-butyltetradecanoic acid, 2-methyloctadecanoic acid, 3-methyloctadecanoic acid, 4-methyloctadecanoic acid, 5-methyloctadecanoic acid, 6-methyloctadecanoic acid, 7-methyloctadecanoic acid, 8-methyloctadecanoic acid, 9-methyloctadecanoic acid, 10-methyloctadecanoic acid, 11-methyloctadecanoic acid, 14-methyloctadecanoic acid, 15-methyloctadecanoic acid, 16-methyloctadecanoic acid, 17-methyloctadecanoic acid, 15-ethylpentadecanoic acid, 3-methylnonadecane acid, 2-ethyloctadecanoic acid, 2-methyleicosanoic acid, 2-propyloctadecanoic acid and 2-butyloctadecanoic acid. Among saturated branched fatty acids, 16-methylheptadecanoic acid (isostearic acid), which has 18 carbon atoms, is preferred.

The esterification product desirably has a degree of esterification of 60% or more because it exerts water-holding property more than lanolin does. If the degree of esterification is less than 60%, the lipophilicity will be poor and the water repellency will be insufficient. If so, the effect of inhibiting the transpiration of moisture from the skin will be reduced and therefore, when being used in a cosmetic, the moisture retention property of the cosmetic will be poor. For this reason, in order to be an esterfication product suitable for use in a cosmetic which improve the moisture retention property to improve skin protection, it is desirable that the degree of esterification is 60% or more.

The degree of esterification is not particularly limited with respect to its upper limit, but it is desirably 90% or less, and preferably 80% or less. This is because when the degree of esterification is more than 90%, the water-holding property will tend to be less than that of lanolin and when it is more than 80%, a water-holding property greater than that of lanolin will be exerted, but the water-holding property will tend to decrease with increase in the degree of esterification.

The acid value of an esterification product is desirably not more than 3.0 because, if so, it is possible to inhibit the esterification product and a cosmetic produced by use of the esterification product from emitting odor. When an esterification product has an acid value more than 3.0, the content of unchanged fatty acid will increase and, therefore, bad effects will occur in the aspect of odor. In addition, it will be feared that skin stimulation be caused by unchanged fatty acid. Therefore, such an esterification product is not suitable as an oily raw material of cosmetics.

The esterification product of branched fatty acid according to the present invention is produced by use of the following method in which the polyglycerol having an average degree of polymerization within a predetermined range and the branched fatty acid having carbon atoms the number of which is within a predetermined range are esterified. First, the branched fatty acid is added to the polyglycerol. In this case, if two or more kinds of branched fatty acids are added, it is only required that predetermined amounts of branched fatty acids are eventually added regardless of whether the branched fatty acids are mixed and then added to the polyglycerol or the branched fatty acids are added to the polyglycerol one after another. Subsequently, an alkaline catalyst such as sodium hydroxide is added to the polyglycerol in which the branched fatty acid was added and then an esterification reaction is carried out under normal or reduced pressure according to a conventional method. It is desirable for the esterification reaction to be carried out continuously until a resulting esterification product comes to have a degree of esterification of 60% or more and an acid value of 3.0 or less.

A cosmetic containing an esterification product of branched fatty acid according to the present invention can be prepared by a conventional method for preparing a cosmetic using the esterification product in the embodiment.

The present invention is explained concretely with reference to Examples, but the present invention is not limited to the Examples.

Esterification products of Examples 1-3 were prepared as follows. Esterification products of Comparative Examples 1-4 were also prepared as references for the esterification products of the Examples. It should be noted that the fatty acids used in the preparations of the esterification products of the Examples and the Comparative Examples were all plant-derived fatty acids.

### EXAMPLE 1

Into a reactor, 100 g of polyglycerol having an average degree of polymerization of 10 calculated on the basis of a hydroxyl value and 337 g of 16-methylheptadecanoic acid (trivial name: isostearic acid) were charged. Following addition of 0.2 g of sodium hydroxide, an esterification reaction was carried out at 250° C. for 4 hours under nitrogen flow. Thus, 395 g of esterification product of branched fatty acid of Example 1 was produced. The esterification product of branched fatty acid had a degree of esterification of 75.0% and an acid value of 1.0.

### EXAMPLE 2

Into a reactor, 100 g of polyglycerol having an average degree of polymerization of 6 calculated on the basis of a hydroxyl value and 150 g of 2-ethylhexanoic acid (trivial name: octylic acid) were charged. Then, a reaction was carried out under conditions the same as those of Example 1. Thus, 220 g of esterification product of branched fatty acid of Example 2 was produced. The esterification product of branched fatty acid had a degree of esterification of 60.0% and an acid value of 0.5.

### EXAMPLE 3

Into a reactor, 100 g of polyglycerol having an average degree of polymerization of 12 calculated on the basis of a hydroxyl value and 351 g of isostearic acid were charged. Then, a reaction was carried out under conditions the same as those of Example 1. Thus, 407 g of esterification product of branched fatty acid of Example 3 was produced. The esterification product of branched fatty acid had a degree of esterification of 80.0% and an acid value of 3.0.

### COMPARATIVE EXAMPLE 1

Into a reactor, 100 g of polyglycerol having an average degree of polymerization of 10 calculated on the basis of a hydroxyl value and 225 g of isostearic acid were charged. Then, a reaction was carried out under conditions the same as those of Example 1. Thus, 295 g of esterification product of branched fatty acid of Comparative Example 1 was produced. The esterification product of branched fatty acid had a degree of esterification of 50.0% and an acid value of 5.0.

### COMPARATIVE EXAMPLE 2

Into a reactor, 100 g of polyglycerol having an average degree of polymerization of 4 calculated on the basis of a hydroxyl value and 380 g of isostearic acid were charged. Then, a reaction was carried out under conditions the same as those of Example 1. Thus, 433 g of esterification product of branched fatty acid of Comparative Example 2 was produced. The esterification product of fatty acid had a degree of esterification of 70.0% and an acid value of 1.0.

### COMPARATIVE EXAMPLE 3

Into a reactor, 100 g of polyglycerol having an average degree of polymerization of 10 calculated on the basis of a hydroxyl value and 335 g of oleic acid were charged. Then, a reaction was carried out under conditions the same as those of Example 1. Thus, 393 g of esterification product of Comparative Example 3 was produced. The esterification product of fatty acid had a degree of esterification of 75.0% and an acid value of 1.0.

### COMPARATIVE EXAMPLE 4

Into a reactor, 100 g of polyglycerol having an average degree of polymerization of 10 calculated on the basis of a hydroxyl value and 337 g of stearic acid were charged. Then, a reaction was carried out under conditions the same as those of Example 1. Thus, 395 g of esterification product of Comparative Example 4 was produced. The esterification product of fatty acid had a degree of esterification of 75.0% and an acid value of 1.0.

The water-holding property and the odor of each of the esterification products of the Examples and the Comparative Examples were examined according to a test of percentage of water hold and a test of odor intensity, each being provided below. Both the tests were carried our also for lanolin and diglyceryl triisostearate.

(Test of Percentage of Water Hold)

A mixture of 1 g of esterification product of an Example, esterification product of a Comparative Example, lanolin or diglyceryl triisostearate and 9 g of vaseline was used as a sample. While the sample was stirred, water was added thereto dropwise slowly. Thus, the sample was caused to hold water until water started oozing. The percentage of water hold of this case was defined by: percentage of water hold (%)=weight of water hold(g)/weight of sample(g)×100.

(Test of Odor Intensity)

On the back of a hand, 1 g of esterification product of an Example, esterification product of a Comparative Example, lanolin or diglyceryl triisostearate was spread and then the odor intensity was evaluated sensorily according to the criteria given below. The number of the monitors who carried out the sensory evaluation was twenty including men and women.
Criteria ◯: Almost no odor is perceived.
   Δ: A slight odor is perceived.
   ×: A strong odor is perceived.

The results of the test of percentage of water hold and the results of the test of odor intensity are shown in Table 1 together with the average degree of polymerizations of the polyglycerols used in the esterifications, the fatty acids, the degrees and acid values of esterification of the esterification products.

From Table 1, it can be confirmed that the esterification products of the Examples each having a degree of esterification of 60% or more and each being obtained by esterification of a polyglycerol having an average degree of polymerization of from 6 to 15 and a branched fatty acid having from 8 to 22 carbon atoms have percentages of water hold which are greater than those of the esterification products of the Comparative Examples and which are higher than that of lanolin by 200% or more.

It can also be confirmed from Table 1 that the esterification product of Comparative Example 1, which has an acid value of 5.0, emits a perceivable odor and the esterification product of Comparative Example 3, which has an acid value of 3.0, also emits a perceivable odor, but the esterification products of all the Examples, which have acid values not more than 3.0, are superior because they emit almost no perceivable odors. In addition, it can also be confirmed from Table 1 that lanolin, which has a percentage of water hold higher than any of the esterification products of Comparative Examples 1-4, emit strongly perceivable odors, but the esterification products of Examples 1-3 of the present invention have unique characteristics in that they have percentages of water hold higher than that of lanolin and emit no perceivable odor.

Separately from the esterification products of Examples 1 to 3, esterification products of Examples 4 to 7 were prepared by use of plant-derived fatty acids as follows.

### EXAMPLE 4

Into a reactor, 100 g of polyglycerol having an average degree of polymerization of 10 calculated on the basis of a hydroxyl value and 337 g of isostearic acid were charged. Then, a reaction was carried out under conditions the same as those of Example 1. Thus, an esterification product of branched fatty acid of Example 4 was produced.

### EXAMPLE 5

An esterification product of branched fatty acid of Example 5 was produced in the same manner as Example 4 except the amount of isostearic acid was changed to 360 g.

### EXAMPLE 6

An esterification product of branched fatty acid of Example 6 was produced in the same manner as Example 4 except the amount of isostearic acid was changed to 405 g.

### EXAMPLE 7

An esterification product of branched fatty acid of Example 7 was produced in the same manner as Example 4 except the amount of isostearic acid was changed to 450 g.

For the resulting esterification products of Examples 4-7, the percentage of water hold was checked in a manner the same as the test of percentage of water hold described above. The results are shown in Table 2.

In Table 2, the percentages of water hold of the esterification products excluding that of Example 7 are higher than the percentage of water hold of lanolin given in Table 1 (360%). In other words, it can be confirmed that the esterification products of Examples 4-7, which are esterification products of the present invention, have water-holding properties and the esterification products of the present invention which have degrees of esterification less than 90% have water-holding properties equivalent to or greater than that of lanolin. In addition, it can be confirmed that if the degree of esterification is greater than 80%, esterification products of the present invention tends to have reduced degrees of water hold.

(Preparation of Cosmetics)

Various types of cosmetics were prepared by compounding the esterification products of Example 1-3 or the Comparative Examples. The preparations of the cosmetics were carried out using the compounding ratios and methods given in the following Formulation Examples 1-9 and Comparative Formulation Examples 1-9.

### FORMULATION EXAMPLE 1

### Emollient Cream

| | (% by weight) |
|---|---|
| Phase A | |
| Esterification product of branched fatty acid of | 5.00 |
| Example 1 | |
| Squalane | 7.50 |
| Decaglyceryl monomyristate | 2.00 |
| Stearic acid | 3.50 |
| Glycerol monostearate | 2.00 |
| Glyceryl tri-2-ethylhexanoic acid | 5.00 |
| Phase B | |
| Glycerol | 7.00 |
| 10 wt % Aqueous solution of potassium hydroxide | 1.00 |
| Purified water | 67.00 |

Phase A was dissolved at 80° C. Then, phase B heated to 80° C. was added thereto slowly to cause emulsification. The emulsification was followed by cooling to 35° C. to yield emollient cream.

### FORMULATION EXAMPLE 2

### Milky Lotion

| | (% by weight) |
|---|---|
| Phase A | |
| Esterification product of branched fatty acid of | 2.50 |
| Example 1 | |
| Decaglyceryl monooleate | 1.00 |
| Phase B | |
| 1 wt % Aqueous solution of carboxyvinyl polymer | 5.00 |
| 10 wt % Aqueous solution of potassium hydroxide | 1.00 |
| 1,3-Butylene glycol | 5.00 |
| Glycerol | 2.00 |
| Purified water | 83.50 |

Phase A was dissolved at 80° C. Then, phase B heated to 80° C. was added thereto slowly to cause emulsification. The emulsification was followed by cooling to 35° C. to yield milky lotion.

### FORMULATION EXAMPLE 3

### Lipstick

| | (% by weight) |
|---|---|
| Phase A | |
| Esterification product of branched fatty acid of | 20.00 |
| Example 2 | |
| Ceresin | 23.50 |
| Castor oil | 27.00 |
| Liquid paraffin | 15.00 |
| Carnauba wax | 7.00 |
| Candelilla wax | 5.00 |
| Phase B | |
| Titanium oxide | 2.00 |
| Red pigment | 0.50 |

Phase A was heated to dissolve homogeneously. The resultant was cooled and then kneaded uniformly with a roll mill. Subsequently, phase B was added, followed by defoaming. The resultant was poured into a mold and cooled rapidly to yield a lipstick.

### FORMULATION EXAMPLE 4

### Shampoo

| | (% by weight) |
|---|---|
| Esterification product of branched fatty acid of | 2.00 |
| Example 2 | |
| Glycerol | 5.00 |
| POE(2) lauryl ether sodium sulfate | 20.00 |
| (27 wt % aqueous solution) | |
| POE(2) lauryl ether sulfuric acid triethanolamine salt | 35.00 |
| (32 wt % aqueous solution) | |
| Polyoxypropylene(36) methyldiethylammonium chlorid | 2.00 |
| Palm kernel fatty acid diethanolamide (1) | 1.00 |
| Sodium chloride | 0.50 |
| Citric acid monohydrate | 0.20 |
| Purified water | 34.30 |

All ingredients were heated to 80° C. to dissolve homogeneously. The resultant was cooled to 35° C. to yield shampoo.

### FORMULATION EXAMPLE 5

### Hair Conditioner

| | (% by weight) |
|---|---|
| Phase A | |
| Esterification product of branched fatty acid of | 3.00 |
| Example 3 | |
| Stearyltrimethylammonium chloride | 0.70 |
| (63 wt % aqueous solution) | |
| Behenyltrimethylammonium chloride | 0.60 |
| (80 wt % aqueous solution) | |
| Stearyl alcohol | 2.50 |
| Lipophilic glycerol monostearate | 0.50 |
| Phase B | |
| Hydroxyethyl cellulose | 0.50 |
| Purified water | 92.20 |

Phase A was dissolved at 80° C. Then, phase B heated to 80° C. was added thereto slowly to cause emulsification. The emulsification was followed by cooling to 35° C. to yield hair conditioner.

### FORMULATION EXAMPLE 6

### Ointment Base

| | (% by weight) |
|---|---|
| Phase A | |
| Esterification product of branched fatty acid of | 10.00 |
| Example 3 | |
| Liquid paraffin | 13.00 |
| Vaseline | 10.00 |
| Cetyl alcohol | 10.00 |
| Decaglyceryl monoisostearate | 3.00 |
| Phase B | |
| Sodium laurylsulfate | 1.00 |
| Purified water | 53.00 |

Phase A was dissolved at 80° C. Then, phase B heated to 80° C. was added thereto slowly to cause emulsification. The emulsification was followed by cooling to 35° C. to yield ointment base.

### FORMULATION EXAMPLE 7

### Cleansing Cream

| | (% by weight) |
|---|---|
| Phase A | |
| Esterification product of branched fatty acid of | 20.00 |
| Example 1 | |
| Squalane | 10.00 |
| Isononanyl isononanoate | 10.00 |
| Cetyl alcohol | 3.00 |
| Lipophilic glycerol monostearate | 3.00 |
| Phase B | |
| Decaglyceryl monoisostearate | 3.00 |
| Glycerol | 5.00 |
| Sodium N-stearoyl-L-glutamate | 1.00 |
| Purified water | 45.00 |

Phase A and phase B were dissolved at 80° C. The phase A was added to the phase B slowly to cause emulsification. The emulsification was followed by cooling to 35° C. to yield cleansing cream.

### FORMULATION EXAMPLE 8

### Hair Wax

| | (% by weight) |
|---|---|
| Phase A | |
| Esterification product of branched fatty acid of | 10.00 |
| Example 2 | |
| Jojoba wax | 3.00 |
| Vaseline | 3.00 |
| Squalane | 2.00 |
| Cetyl alcohol | 1.50 |
| Self-emulsifying glycerol monostearate | 2.00 |
| Phase B | |
| Diglycerol | 5.00 |
| Decaglyceryl monomyristate | 2.00 |
| 2 wt % Aqueous solution of acrylic acid-alkyl | 18.00 |
| methacrylate copolymer | |
| 2 wt % Aqueous solution of carboxyvinyl polymer | 5.00 |
| 10 wt % Aqueous solution of potassium hydroxide | 1.80 |
| Purified water | 46.70 |

Phase A and phase B were dissolved at 80° C. The phase A was added to the phase B slowly to cause emulsification. The emulsification was followed by cooling to 35° C. to yield hair wax.

### FORMULATION EXAMPLE 9

### Oil-Based Foundation (Stick Type)

| | (% by weight) |
|---|---|
| Phase A | |
| Esterification product of branched fatty acid of | 15.00 |
| Example 3 | |
| Solid paraffin | 7.50 |
| Microcrystalline wax | 7.00 |
| Diisostearyl malate | 5.00 |
| Phytosteryl oleate | 5.00 |
| Purified water | 10.00 |
| Phase B | |
| Kaolin | 23.00 |
| Titanium oxide | 23.00 |
| Red iron oxide | 1.00 |
| Yellow iron oxide | 3.00 |
| Black iron oxide | 0.50 |

Phase A was heated to 85° C. to dissolve homogeneously. To the resultant, phase B, which had been mixed and pulverized, was added under stirring, followed by grinding dispersion in a colloid mill. After degassing, the resultant was poured into a mold at 70° C. and cooled rapidly to yield oil-based foundation.

### COMPARATIVE FORMULATION EXAMPLE 1

### Emollient Cream

| | (% by weight) |
|---|---|
| Phase A | |
| Esterification product of Comparative Example 1 | 5.00 |
| Squalane | 7.50 |
| Decaglyceryl monomyristate | 2.00 |
| Stearic acid | 3.50 |
| Glycerol monostearate | 2.00 |
| Glyceryl tri-2-ethylhexanoic acid | 5.00 |
| Phase B | |
| Glycerol | 7.00 |
| 10 wt % Aqueous solution of potassium hydroxide | 1.00 |
| Purified water | 67.00 |

Phase A was dissolved at 80° C. Then, phase B heated to 80° C. was added thereto slowly to cause emulsification. The emulsification was followed by cooling to 35° C. to yield emollient cream.

### COMPARATIVE FORMULATION EXAMPLE 2

### Milky Lotion

| | (% by weight) |
|---|---|
| Phase A | |
| Esterification product of Comparative Example 2 | 2.50 |
| Decaglyceryl monooleate | 1.00 |
| Phase B | |
| 1 wt % Aqueous solution of carboxyvinyl polymer | 5.00 |
| 10 wt % Aqueous solution of potassium hydroxide | 1.00 |
| 1,3-Butylene glycol | 5.00 |
| Glycerol | 2.00 |
| Purified water | 83.50 |

Phase A was dissolved at 80° C. Then, phase B heated to 80° C. was added thereto slowly to cause emulsification. The emulsification was followed by cooling to 35° C. to yield milky lotion.

### COMPARATIVE FORMULATION EXAMPLE 3

### Lipstick

| | (% by weight) |
|---|---|
| Phase A | |
| Esterification product of Comparative Example 3 | 20.00 |
| Ceresin | 23.50 |
| Castor oil | 27.00 |
| Liquid paraffin | 15.00 |
| Carnauba wax | 7.00 |
| Candelilla wax | 5.00 |
| Phase B | |
| Titanium oxide | 2.00 |
| Red pigment | 0.50 |

Phase A was heated at 80° C. to dissolve homogeneously. The resultant was cooled and then kneaded uniformly with a roll mill. Phase B was added thereto, followed by defoaming. The resultant was poured into a mold and cooled rapidly to yield a lipstick.

### COMPARATIVE FORMULATION EXAMPLE 4

### Shampoo

| | (% by weight) |
|---|---|
| Esterification product of Comparative Example 4 | 2.00 |
| Glycerol | 5.00 |
| POE(2) lauryl ether sodium sulfate (27 wt % | 20.00 |
| aqueous solution) | |
| POE(2) lauryl ether sulfuric acid triethanolamine | 35.00 |
| salt (32 wt % aqueous solution) | |
| Polyoxypropylene(36) methyldiethylammonium | 2.00 |
| chloride | |
| Palm kernel fatty acid diethanolamide (1) | 1.00 |
| Sodium chloride | 0.50 |
| Citric acid monohydrate | 0.20 |
| Purified water | 34.30 |

All ingredients were heated to 80° C. to dissolve homogeneously. The resultant was cooled to 35° C. to yield shampoo.

### COMPARATIVE FORMULATION EXAMPLE 5

### Hair Conditioner

| | (% by weight) |
|---|---|
| Phase A | |
| Esterification product of Comparative Example 1 | 3.00 |
| Stearyltrimethylammonium chloride (63 wt % | 0.70 |
| aqueous solution) | |
| Behenyltrimethylammonium chloride (80 wt % | 0.60 |
| aqueous solution) | |
| Stearyl alcohol | 2.50 |
| Lipophilic glycerol monostearate | 0.50 |
| Phase B | |
| Hydroxyethyl cellulose | 0.50 |
| Purified water | 92.20 |

Phase A was dissolved at 80° C. Then, phase B heated to 80° C. was added thereto slowly to cause emulsification. The emulsification was followed by cooling to 35° C. to yield hair conditioner.

### COMPARATIVE FORMULATION EXAMPLE 6

### Ointment Base

| | (% by weight) |
|---|---|
| Phase A | |
| Esterification product of Comparative Example 2 | 10.00 |
| Liquid paraffin | 13.00 |
| Vaseline | 10.00 |
| Cetyl alcohol | 10.00 |
| Decaglyceryl monomyristate | 3.00 |
| Phase B | |
| Sodium laurylsulfate | 1.00 |
| Purified water | 53.00 |

Phase A was dissolved at 80° C. Then, phase B heated to 80° C. was added thereto slowly to cause emulsification. The emulsification was followed by cooling to 35° C. to yield ointment base.

### COMPARATIVE FORMULATION EXAMPLE 7

### Cleansing Cream

| | (% by weight) |
|---|---|
| Phase A | |
| Esterification product of Comparative Example 3 | 20.00 |
| Squalane | 10.00 |
| Isononanyl isononanoate | 10.00 |
| Cetyl alcohol | 3.00 |
| Lipophilic glycerol monostearate | 3.00 |
| Phase B | |
| Decaglyceryl monolaurate | 3.00 |
| Glycerol | 5.00 |
| Sodium N-stearoyl-L-glutamate | 1.00 |
| Purified water | 45.00 |

Phase A and phase B were dissolved at 80° C. The phase A was added to the phase B slowly to cause emulsification. The emulsification was followed by cooling to 35° C. to yield cleansing cream.

### COMPARATIVE FORMULATION EXAMPLE 8

### Hair Wax

| | (% by weight) |
|---|---|
| Phase A | |
| Esterification product of Comparative Example 4 | 10.00 |
| Jojoba wax | 3.00 |
| Vaseline | 3.00 |
| Squalane | 2.00 |
| Cetyl alcohol | 1.50 |
| Self-emulsifying glycerol monostearate | 2.00 |
| Phase B | |
| Diglycerol | 5.00 |
| Decaglyceryl monomyristate | 2.00 |
| 2 wt % Aqueous solution of acrylic acid-alkyl | 18.00 |
| methacrylate copolymer | |
| 2 wt % Aqueous solution of carboxyvinyl polymer | 5.00 |
| 10 wt % Aqueous solution of potassium hydroxide | 1.80 |
| Purified water | 46.70 |

Phase A and phase B were dissolved at 80° C. The phase A was added to the phase B slowly to cause emulsification. The emulsification was followed by cooling to 35° C. to yield hair wax.

### COMPARATIVE FORMULATION EXAMPLE 9

### Oil-Based Foundation (Stick Type)

| | (% by weight) |
|---|---|
| Phase A | |
| Esterification product of Comparative Example 1 | 15.00 |
| Solid paraffin | 7.50 |
| Microcrystalline wax | 7.00 |
| Diisostearyl malate | 5.00 |
| Phytosteryl oleate | 5.00 |
| Purified water | 10.00 |
| Phase B | |
| Kaolin | 23.00 |
| Titanium oxide | 23.00 |
| Red iron oxide | 1.00 |
| Yellow iron oxide | 3.00 |
| Black iron oxide | 0.50 |

Phase A was heated to 85° C. to dissolve homogeneously. To the resultant, phase B, which had been mixed and pulverized, was added under stirring, followed by grinding dispersion in a colloid mill. After degassing, the resultant was poured into a mold at 70° C. and cooled rapidly to yield oil-based foundation.

Using the cosmetics of the Formulation Examples and Comparative Formulation Examples, evaluations of moisture retention property (wet feeling), oily feeling such as stickiness and odor were carried out. These evaluations were conducted through direct comparisons of the emollient cream of Formulation Example 1 to that of Comparative Formulation Example 1, of the milky lotion of Formulation Example 2 to that of Comparative Formulation Example 2, of the lipstick of Formulation Example 3 to that of Comparative Formulation Example 3, of the shampoo of Formulation Example 4 to that of Comparative Formulation Example 4, of the hair conditioner of Formulation Example 5 to that of Comparative Formulation Example 5, of the ointment base of Formulation Example 6 to that of Comparative Formulation Example 6, of the cleansing cream of Formulation Example 7 to that of Comparative Formulation Example 7, of the hair wax of Formulation Example 8 to that of Comparative Formulation Example 8, and of the oil-based foundation of Formulation Example 9 to that of Comparative Formulation Example 9.

In every direct comparison of a formulation example to a comparative formulation example, it was confirmed that a cosmetic of a comparative formulation example was sensorily unsatisfactory because of insufficient wet feeling and stickingly oily feeling, whereas a cosmetic of a formulation example was superior in wet feeling and offered smooth feeling in use without offering oily feeling. The cosmetics of Comparative Formulation Examples 1, 3, 5, 7 and 9, which were prepared using the odor-emitting esterification products of Comparative Example 1 or 3, emitted odor. As opposed to this, not only the cosmetics of Formulation Examples 1, 3, 5, 7 and 9 but the cosmetics of all the Formulation Examples, which were prepared using the esterification products of the present invention, emitted almost no annoying odor and were products which can be used satisfactorily as unscented cosmetics.

### INDUSTRIAL APPLICABILITY

From presently invented esterification products of branched fatty acids, cosmetic having high moisture retention properties can be produced when the esterification products are used as oily raw materials of cosmetics. The esterification products of the present invention can be used as raw materials of various cosmetics such as skin care cosmetics, i.e. cream and lotion; makeup cosmetics, i.e. lipstick and foundation; hair cosmetics, i.e. shampoo, conditioner and hair wax; cleansing cosmetics; and ointments.

TABLE 1

| | Average degree of | | | | Percentage | |
|---|---|---|---|---|---|---|
| | polymerization of | | Degree of | Acid | of water | |
| Sample | polyglycerol | Fatty acid | esterification (%) | value | hold (%) | Odor |
| Example 1 | 10 | Isostearic acid | 75.0 | 1.0 | 560 | ∘ |
| Example 2 | 6 | Octylic acid | 60.0 | 0.5 | 510 | ∘ |
| Example 3 | 12 | Isostearic acid | 80.0 | 3.0 | 580 | ∘ |
| Comparative | 10 | Isostearic acid | 50.0 | 5.0 | 330 | x |
| Example 1 | | | | | | |
| Comparative | 4 | Isostearic acid | 70.0 | 1.0 | 200 | ∘ |
| Example 2 | | | | | | |
| Comparative | 10 | Oleic acid | 75.0 | 1.0 | 300 | Δ |
| Example 3 | | | | | | |
| Comparative | 10 | Stearic acid | 75.0 | 1.0 | 180 | ∘ |
| Example 4 | | | | | | |
| Lanolin | — | — | — | — | 360 | x |
| Diglyceryl | — | — | 75.0 | 1.0 | 290 | ∘ |
| triisostearate | | | | | | |

TABLE 2

| | Average degree of | | Degree of | Percentage of |
|---|---|---|---|---|
| | polymerization | | esterifica- | water hold |
| Sample | of polyglycerol | Fatty acid | tion (%) | (%) |
| Example 4 | 10 | Isostearic | 75 | 540 |
| | | acid | | |
| Example 5 | 10 | Isostearic | 80 | 500 |
| | | acid | | |
| Example 6 | 10 | Isostearic | 90 | 360 |
| | | acid | | |
| Example 7 | 10 | Isostearic | 100 | 230 |
| | | acid | | |

1. An esterification product which is an esterification product obtained by esterifying polyglycerol and a fatty acid, wherein the polyglycerol has an average degree of polymerization of from 6 to 15 calculated on the basis of the hydroxyl value thereof, the fatty acid is at least one branched fatty acid selected from branched fatty acids having 8 to 22 carbon atoms, the esterification product has a degree of esterification of 60% or more.

2. The esterification product according to claim 1 wherein the esterification product has an acid value of 3.0 or less.

3. A cosmetic which includes the esterification product according to claim 1.

4. A cosmetic which includes the esterification product according to claim 2.
